(19) United States
(12) Patent Application Publication
(10) Pub. No.: US 2009/0325219 A1
(43) Pub. Date: Dec. 31, 2009

(54) DEVICE, SYSTEM AND ASSAY FOR MEASURING CELL MOTILITY

(75) Inventors: Kate Williamson, Belfast (GB); Vincent Koo, Belfast (GB); Perry Maxwell, Belfast (GB)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: The Queen's University of Belfast, Belfast (GB)

(21) Appl. No.: 12/310,098

(22) PCT Filed: Aug. 9, 2007
(86) PCT No.: PCT/GB2007/003052

§ 371 Date: Jun. 12, 2009

(30) Foreign Application Priority Data
Aug. 9, 2006 (GB) 0615813.3

(51) Int. Cl. C12Q 1/24; C12M 1/34

(52) U.S. Cl. 435/30; 435/287.1; 435/287.3

(57) ABSTRACT

The present application relates to a device and system for measuring cell motility comprising a supporting substrate which includes at least one cell receiving zone and an agent receiving matrix provided on a matrix receiving zone, wherein interposed between the cell receiving zone and the agent receiving matrix is a bridging portion. In addition, methods of using said device and system and a kit relating to the device is provided.

### ASSAY

The present invention relates to a device for measuring cell motility or migration and a method for monitoring cell migration. In particular, the present invention relates to an assay and method for monitoring and isolating cells.

### BACKGROUND

The acquisition of invasive potential by a cell is a watershed in tumourigenesis marking the ability of a tumour cell to spread. An understanding of cancer progression and eventual metastasis is crucial to aid the development of novel treatment strategies and this is reflected by a large number of assays in use in current research studies that involve the assessment of metastatic potential.

Cell migration is also important in understanding the inflammatory response, as migration of leukocytes to a damaged area to assist in fighting infection or healing the wound is critical. In addition, cell migration is of importance in understanding angiogenesis.

Objective in vitro cell motility measurement techniques currently in use include scratch wound assays which assess cell motility (Lampugnani M G. Cell migration into a wounded area in vitro. Methods Mol Biol 1999, 96:177-182); matrigel invasive assays which measure cell invasion through a reconstituted basement membrane (Albini A, Iwamoto Y, Kleinman H K, Martin G R, Aaronson S A, Kozlowski J M, McEwan R N. A rapid in vitro assay for quantitating the invasive potential of tumor cells. Cancer Res. 1987 Jun. 15; 47(12):3239-45); 3D-culture assays (Hurst R E, Kyker K D, Bonner R B, Bowditch R D, Hemstreet G P, 3rd. Matrix-dependent plasticity of the malignant phenotype of bladder cancer cells. Anticancer Res. 2003 Jul.-Aug.; 23(4):3119-3128.; the soft agar assay which measures anchorage-independent cell proliferation (Fukazawa H, Mizuno S, Uehara Y. A microplate assay for quantitation of anchorage-independent growth of transformed cells. Anal Biochem. 1995 Jun. 10; 228(1):83-90) and cell adhesion assays which correlate tumourigenicity with cell adhesiveness (Moye V E, Barraclough R, West C, Rudland P S. Osteopontin expression correlates with adhesive and metastatic potential in metastasis-inducing DNA-transfected rat mammary cell lines. Br J. Cancer. 2004 May 4; 90(9):1796-802).

Typically, current assays used to assess motility/migratory potential of a cell population are subjective and may only measure one aspect of the factors which in combination are important in characterising invasive potential.

The scratch wound assay creates damaged cells as a result of scratching a monolayer of cells to produce a wound and then assesses the rate and extent of wound closure as a measure of cell motility. A disadvantage of the scratch wound assay is the wound created in the scratch wound assay releases several potent growth inhibitors and promoters that can affect cell migration and subsequent closure of the wound (Tsuboi K, Yamaoka S, Maki M, Ohshlo G, Tobe T, Hatanaka M. Soluble factors including proteinases released from damaged cells may trigger the wound healing process. Biochem Biophys Res Commun. 1990 May 16; 168(3):1163-70).

Matrigel and Boyden chamber assays suffer from the disadvantage that preliminary studies need to be carried out on the cells being investigated to determine the doubling time of the cells. This is due to the possibility that cells which have successfully migrated across the substitute basement membrane pores into the serum-rich media could themselves begin to divide and proliferate. A second shortcoming is that the Boyden chamber requires destruction of cells for analysis and therefore precludes kinetic analysis using video-microscopy.

Furthermore, in both the scratch wound and Matrigel assays it is not possible to accurately separate out tumour cell populations into distinct sub populations of motile and non motile components which can subsequently be characterised to elucidate important differences between the sub-populations.

### SUMMARY OF THE INVENTION

The inventors have developed a novel assay method and device to perform the same that provides an accurate assessment of cell migration, particularly cancer cell migration, towards or away from an agent, for example, towards a chemo-attractant or a chemo-inhibitory agent. This assay has the added advantage that it may be used to facilitate characterisation of an aggressive sub-population of cells with respect to protein or nucleic acid profiles hence facilitating the identification of novel markers of metastasis.

According to a first aspect of the present invention there is provided a device for measuring cell motility comprising a supporting substrate which includes at least one cell receiving zone and an agent receiving matrix provided on a matrix receiving zone, wherein interposed between the cell receiving zone and the agent receiving matrix is a bridging portion.

In use, a cell moving from the cell receiving zone to the matrix provided on the matrix receiving zone has to cross the bridging portion. The cell receiving portion and the agent receiving matrix is separated by fluid media which fluidly connects the cell receiving portion and the agent receiving matrix and enables the cells located in the cell receiving zone to come into contact with agent provided in the agent receiving matrix.

In preferred embodiments the bridging portion is a portion of the supporting substrate. However, as will be appreciated, the bridging portion can be provided by a film, coating or other material, located on the substrate, on which the cells can locomote. For example the supporting substrate may or may not be coated with substances such as type IV collagen, laminin, fibronectin, vitronectin or other substances representative of the extracellular matrix.

Suitably, in particular embodiments, the agent receiving matrix substantially surrounds the cell receiving zone.

Suitably, in particular embodiments, the supporting substrate is a coated or uncoated cell culture plate comprising at least one vessel or well wherein said vessel or well has a base and at least one sidewall defining an opening, wherein the base of the vessel or well includes the cell receiving zone and the matrix receiving zone. The vessel or well is not limited to any particular cross section and can be hexagonal, circular, semi-circular, ellipsoidal, rectangular, square or any other polygonal or curved shape.

In particular embodiments the cell supporting substrate is a cell culture plate comprising a plurality of vessels or wells, for example a multi-well tissue plate.

An advantage of the device of the present invention is that it provides for real-time observation of cell motility and, for observation, the cells do not require to be killed.

This is advantageous as motile cells which move from the cell receiving zone over the bridging portion towards the agent receiving matrix can be distinguished from those cells which remain in the cell receiving zone.

In particular embodiments of the device, the cell receiving zone can be of width in the range 5 mm to 20 mm, preferably about 10 mm. In an embodiment the matrix receiving zone, and in turn the matrix, can be of width in the range 5 mm to 20 mm, preferably about 10 mm.

In particular embodiments the distance of the bridging portion between the cell receiving zone and the matrix receiving zone can range from 2 mm to 10 mm. In preferred embodiments of the device the bridging portion can be about 2 mm. Advantageously, such dimensions mean that cells have to travel a sufficient distance from the cell receiving zone to the matrix receiving zone that motile cells can be distinguished from other cells in the sample. In addition, it may be possible to observe direction of motile cells, for example towards an agent, for example, chemotaxis, haptotaxis or chemoinvasion or away from an agent.

In particularly preferred embodiments the supporting substrate can be formed from a material which is compatible with cells. Suitable materials can include, glass, ceramics, metals, plastics, polymers including, but not limited to polystyrene, polycarbonate, polypropylene or polymeric thin films.

The supporting substrate may comprise a substance on its surface which aids location of a sample, for example a biological or cell sample, which aids adhesion or movement of cells or which mimics in vivo conditions. In particular embodiments the substrate can comprise biological material including proteins or cells. Additionally or alternatively the substrate can include patterns on its surface.

Suitably the cell receiving zone may be an indentation provided in the supporting substrate which is adapted to receive a cell support means. The cell support means is a support on which cells can be grown and/or a sample can be mounted and then the sample provided to the device.

In particular embodiments the cell support means can be a microscope slide or coverslip. In such embodiments, in use, cells can be placed or grown on the cell support means and the cell support means provided into a suitably shaped and sized indentation in the supporting substrate such that a top surface of the cell support means, when the cell support means is located in the indentation, is substantially in the same plane as the receiving surface of the cell receiving-zone around the cell support.

In an embodiment of the device including an indentation in the surface of the receiving zone, said indentation may be in the range 0.1 to 0.3 mm in depth from the surface of the supporting substrate.

It is advantageous for the device to include means for locating cell support means as it allows a sample to be provided to the device, motility of cells of the sample to be determined, and the cells of the sample remaining in the cell receiving zone to be removed such that the motile and non-motile cells can be isolated from each other. This provides for differential screening of migratory versus static sub populations of cells being studied.

Suitably the indentation may be about 0.15 mm in depth from the surface of the supporting substrate.

In use, to objectively determine if cells have moved from the cell receiving zone, it is advantageous to provide means of measuring movement of a cell. In particular embodiments of the device, the means for measuring movement of a cell can include at least one marking around the cell receiving zone or spaced apart from the cell receiving zone to allow accurate quantification of movement of cells from a sample located within the cell receiving zone. Preferably a plurality of markings are provided wherein said markings are spaced apart from each other. More preferably, said markings can be arranged as a concentric grid emanating from the edge of the cell receiving zone towards the matrix receiving zone.

In embodiments of the device wherein the cell receiving zone includes an indentation, said markings may be arranged as a concentric grid emanating from the edge of the indentation and extending towards the matrix receiving zone.

In particular embodiments of the device, the supporting substrate may be optically clear and include on or in the supporting substrate a scoring grid or markings spaced apart from each other.

Suitably the markings and/or scoring grid may be located under at least part of the cell receiving zone and the matrix receiving zone.

The provision of markings and/or a scoring grid is advantageous as it allows an objective measure of the motility of cells.

An advantage of the device of the present invention is that, in use, those cells which are motile are easily distinguished from those which are not or those which are less motile. As explained above in embodiments of the device comprising a cell support means, said populations of cells can be easily isolated from each other using the device.

Suitably, in particular embodiments of the device, the agent receiving matrix is a matrix of reproducible dimensions. This is advantageous as this aspect can then be standardised. Suitably the matrix can be formed from, but is not limited to, agarose, agar, or collagen.

The agent provided to the agent receiving matrix can comprise molecules, for example biomolecules, known to affect chemotaxis or haptotaxis (movement of cells in response to a concentration gradient of a substrate-bound stimulus). These can include, but are not limited to, DNA, RNA, proteins, peptides, carbohydrates, cells, for example cancer cells, biochemicals, or small molecules.

In particular embodiments, agents can include chemo-attractant agents, which can include, but are not limited to; fetal calf serum, autologous serum conditioned media, cytokines or chemokines, growth factors, EGF, bFGF, NGF, PDGF, IGF-1, TGF-β, nutrients, small molecules, hormones or the like. The assay has a further potential benefit of controlled release of soluble compounds from the agarose.

As will be appreciated by those of skill in the art, the components of the device may be provided as component parts. Accordingly, a second aspect of the present invention provides a system for determining cell motility comprising:

i) a device comprising a supporting substrate which includes at least one cell receiving zone and a matrix receiving zone, wherein interposed between the cell receiving zone and the matrix receiving zone is a bridging portion; and
ii) an agent receiving matrix.

In particular embodiments of the system a plurality of agent receiving matrices can be provided which can be located in/on the matrix receiving zone. Respective matrices can comprise particular agents, particular combinations of agents or particular concentrations of agents. As will be appreciated a selection of a matrices is advantageous as it provides for a standard test device which can be used with different cell samples, different agent containing matrices and/or different concentrations of an agent in a matrix as part of the system.

Suitably, the system of the present invention can include imaging means, for example a video camera, a phase contrast microscope, a confocal microscope, a luminescence detector, a fluorescence detector or the like to allow detection of the rate or extent of motility of cells provided to the device.

In particular embodiments of the system, the means for measuring movement of a cell from the cell receiving zone may be provided on a microscope used to view the cells.

In preferred embodiments, the device is provided with the same footprint/dimensions as a standard multiwell culture plate wherein the cell receiving zone and the matrix receiving zone is provided by the base of the respective well such that the system can include standard fluid handling means. This is advantageous as it allows high throughput screening methods to be applied to the device. In particular embodiments, the system can include robotic fluid handling and automated detection of cell motility.

In particular embodiments of the device of the system, the supporting substrate can be manufactured and a suitable matrix then added subsequent to the manufacture of the supporting substrate. In such embodiments the matrix may be provided using a suitably shaped mould.

Accordingly, a third aspect of the present invention provides a kit for forming the device of the present invention comprising:
a supporting substrate which includes at least one cell receiving zone and a matrix receiving zone, wherein interposed between the cell receiving zone and the matrix receiving zone is a bridging portion; and
an agent receiving matrix forming mould.

In particular embodiments the mould may comprise a first sidewall, a second sidewall and a matrix receiving channel interposed between said sidewalls. Suitably the mould can be circular in shape.

In other embodiments the mould can be circular in shape and comprise one circumferential sidewall which defines the perimeter of the matrix to be formed. In use, at least one aperture may be provided to the moulded matrix such that said matrix may be positioned around a cell receiving zone and access to the cell receiving zone is provided.

In such embodiments, the kit can include a cell receiving zone cut out member, wherein said member has a matrix cutting edge wherein, in use, the cylinder is placed into a formed matrix to cut out a portion of the matrix. In preferred embodiments, the member is cylindrical in shape, with a diameter such that a sample located in the cut out portion, placed over the cell receiving zone is spaced apart from the matrix such that a portion of the supporting substrate around the cell receiving zone forms a bridging portion between the cell receiving zone and the matrix receiving zone.

In particular embodiments the member has a diameter in the range 5 mm to 25 mm.

In use, the device of the present invention may be used to measure cell motility.

According to a fourth aspect of the present invention there is provided an assay method using a device or system of the present invention comprising the steps:
providing a sample of cells to a cell receiving zone, such that the sample of cells is in fluid connection with the agent receiving matrix, and
determining movement of cells provided in the sample from the cell receiving zone relative to the agent receiving matrix.

In one embodiment, the sample of cells is provided in tissue culture media. In another embodiment tissue culture media is added to the sample of cells in the cell receiving zone. In particular embodiments of the invention, the fluid connection between the sample cell receiving zone and the agent receiving matrix is provided by tissue culture media.

In particular embodiments, an agent(s) can be provided to the matrix to determine the influence the agent(s) have on the movement of cells.

In particular embodiments, the sample can comprise cells selected from the group consisting of lymphocytes, monocytes, leukocytes, macrophages, mast cells, T-cells, B-cells, neutrophils, basophils, eosinophils, fibroblasts, endothelial cells, epithelial cells, neurons, tumour cells, motile gametes, bacteria, fungi, cells involved in inflammation, cells involved in angiogenesis, cells involved in response to injury, or cells involved in response to infection.

The assay method allows the effect on a cell of an agent, or the concentration of an agent provided to the agent receiving matrix with respect to cell motility of said cell, to be determined. As will be appreciated by those of skill in the art, different agents or different concentrations of the same agent may be objectively compared using the assay method of the present invention.

In particular embodiments, the method can comprise at least one additional step selected from characterisation of motile cells moving away from the cell receiving zone, isolation of motile cells moving away from the cell receiving zone, fixation for immunocytochemistry of motile cells moving away from the cell receiving zone.

Alternatively, or additionally, in particular embodiments, the method can include at least one additional step selected from characterisation of non-motile cells which do not move away from the cell receiving zone, isolation of non-motile cells which do not move away from the cell receiving zone, fixation for immunocytochemistry of non-motile cells which do not move away from the cell receiving zone.

Characterisation can include determination of nucleic acid expression and/or protein profiles in a cell.

One key advantage of this novel assay is that it allows characterisation of cells constituting distinct populations or sub-populations capable of migration. At least three sub-populations could be characterised: static, motile and migratory cells. Comparison of the profiles of motile cells which move from the cell receiving zone and the profiles of cells that remain in the cell receiving zone allows determination of the differences of characteristics between the two populations. Cells which migrate into the agent receiving matrix could also be harvested for characterisation, for example nucleic acid evaluation or for morphological comparison. Typically, conventional assays are not able to accurately discriminate between such populations. As will be appreciated by those of skill in the art, determinations of those cells which have moved from the cell receiving zone may be performed over defined time periods to determine cells with particular motilities.

In circumstances where cells from the sample of cells move from the cell receiving zone towards the agent receiving zone at different rates, distinct sub-populations of cells may be provided.

Suitably, in particular embodiments, the step of characterisation can include
a step of differential staining of cells with distinct profiles,
a step of harvesting motile and/or non motile cell populations and/or sub-populations, or
a step of profiling protein and/or nucleic acid expressed by particular cells.

Suitably, in particular embodiments, the method can comprise the step of providing a sample on a cell support means, for example a coverslip, and locating the cell support means, for example the coverslip, in the cell receiving zone of the supporting substrate.

Suitably, in particular embodiments, the sample can be a tissue sample, for example a tissue explant. This may be advantageous as it allows the assay to be applied to a tumour explant from a patient to determine whether a tumour cell of the explant has a metastatic phenotype.

In particular embodiments of the method, following removal of tissue from a cancer patient(s), a representative tumour explant(s) may be grown on a cell support means and the cell support means can be located in the cell receiving zone of the device. The extent of migration of the cells of the sample can be determined using the assay method of the present invention.

Advantageously the assay method of the present invention provides a diagnostic test to determine the metastatic potential of individual tumour samples.

Accordingly, a fifth aspect of the present invention provides a method of using the device or system of the invention to determine the motility of one or more cells of an individual tumour of an individual patient comprising the steps:
providing an explant derived from tissue obtained from a cancer patient to the cell receiving zone, such that cells of the explant are in fluid connection with the agent receiving matrix, and
determining movement of cells provided in the sample from the cell receiving zone relative to the agent receiving matrix.

The method can include the step of providing a test agent to the agent receiving matrix.

The movement of cells from the cell receiving zone towards the agent receiving matrix may be used to measure the cells' motility in response to different agents or concentrations of an agent provided to the agent receiving matrix, and thus the cells' metastatic potential.

To determine metastatic potential of cells obtained from a cancer patient the method may further include a step of determining the ability of cells to express indicators of invasion potential. The results from this assay method can allow prediction of those patients at risk of metastasis and may allow for the stratification of patients into distinct treatment regimens.

The present assay may be used to objectively compare the effect of different test agents on given samples of cells.

Accordingly, a sixth aspect of the present invention provides a method to determine the ability of a test agent to modulate cell motility, said method comprising the steps:
providing a sample of cells to a device according to the first aspect of the invention or a system according to the second aspect of the invention, such that the sample is in fluid connection with the agent receiving matrix, and
determining movement of cells provided in the sample from the cell receiving zone relative to the agent receiving matrix in the presence of a test agent in the matrix relative to movement in the absence of said test agent.

In one embodiment, decreased movement of cells (for example, slower movement and/or decreased distance traveled) in the presence of test agent compared to in its absence is indicative that the test agent is an inhibitor of cell motility. In one embodiment, the identification of a test agent as an inhibitor of cell motility is indicative that it is anti-metastatic.

In another embodiment, enhanced movement of cells (for example, faster movement and/or increased distance traveled) in the presence of test agent compared to in its absence is indicative that the test agent is an enhancer of cell motility. In one embodiment, the identification of a test agent as an enhancer is indicative that it is pro-metastatic.

In one embodiment of this method, the effect of the test agent on the movement of cells is determined by (i) determining the movement of cells provided in the sample from the cell receiving zone relative to the agent receiving matrix in the absence of the test agent and then, (ii) adding the test agent to the agent receiving matrix and determining the movement of cells in the presence of the test agent.

In another embodiment, parallel tests may be performed in which two samples are provided under the same conditions in identical devices, with the test agent provided to the matrix of a first device, but not to the matrix of the second device. The effect of the test agent may then be determined by comparing the movement of cells in the device, in which the test agent is provided with the movement of cells in the device in which the test agent is absent.

In one embodiment of the invention, where the test agent is absent, a control agent may be used.

Suitably, in particular embodiments, the method of the invention can employ a plurality of different test agents and/or a plurality of different concentrations of a test agent and the extent to which each test agent inhibits cell motility can be assessed.

The method to determine a test agent which inhibits cell motility can be used to determine therapeutic agents that inhibit the progress of inflammatory diseases, for example arthritis, skin diseases and the like. It can additionally or alternatively be used to determine agents important in angiogenesis, for example agents which block blood vessel growth or improve vessel function. Further, the method may be used to test for agents which can affect one or more of cancer, tissue regeneration, organ transplantation, autoimmune diseases and other degenerative diseases.

Where an agent provided to the matrix slows the motility of cells of the sample and the sample comprises cancer cells, said agent may be an anti-cancer agent. In particular embodiments, the agent can be anti-metastatic.

Preferred features and embodiments of each aspect of the invention are as for each of the other aspects mutatis mutandis unless context demands otherwise.

Embodiments of the device and methods of the present invention will now be provided, by way of example only, with reference to the following figures wherein:

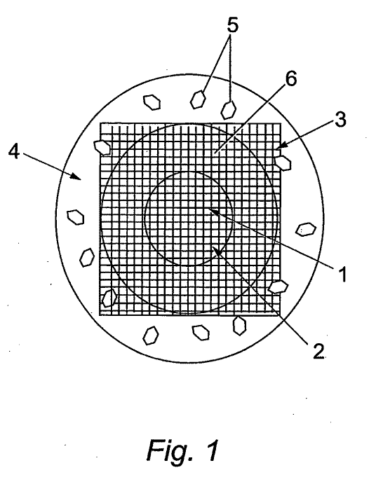

FIG. 1 is an illustration of an embodiment of the device of the present invention wherein the device includes a cell receiving zone (1) for receiving cells comprising an indentation, wherein the indentation is suitably sized and shaped to locate cell support means (2), illustrated as a coverslip, such that the surface of the coverslip on which the sample is located is flush with the surface of the surrounding substrate of the cell receiving zone, a grid under the surface of the substrate for objective counting purposes (3), an agent receiving zone (4) comprising at least one agent (5) or concentration of agent and a bridging portion (6) interposed between the cell receiving zone (1) and the agent receiving zone (4);

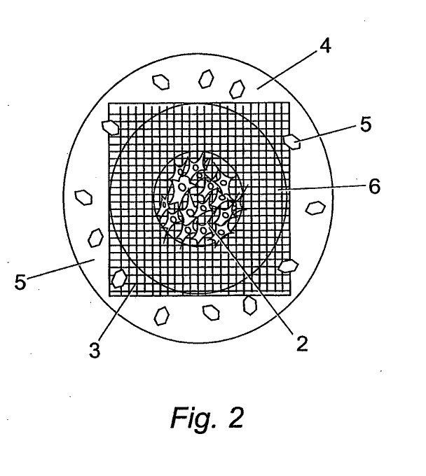

FIG. 2 is an illustration of the embodiment of the device of FIG. 1 when cells have been located in the cell receiving zone by a user locating a cell support means (2) (coverslip) with cells of interest at 100% confluency in the indentation;

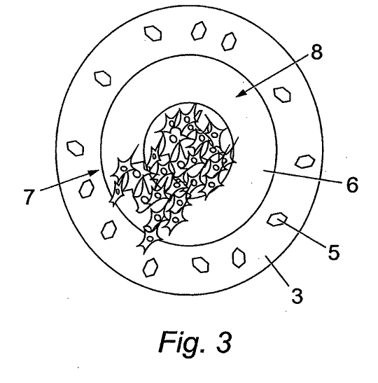

FIG. 3 is an illustration of the embodiment of the device of FIG. 1 wherein a population of the sample of cells placed in the cell receiving zone have moved towards the agent zone (motile cells (7)) whereas non-motile cells (8) remain in the cell receiving zone;

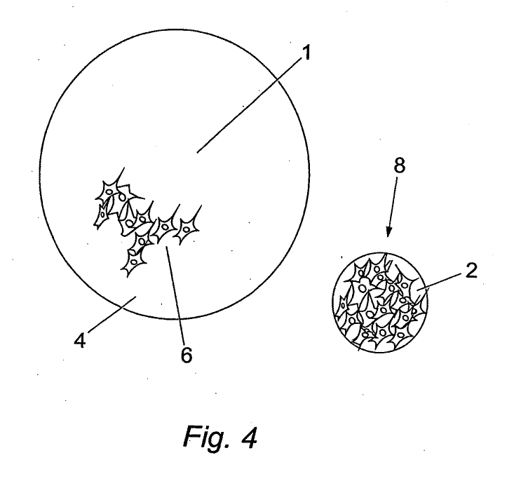

FIG. 4 illustrates the manipulation (isolation) of the population of the cell sample which moved from the cell receiving area towards the agent receiving zone and indicates the way in which distinct populations can be isolated for differential immunostaining;

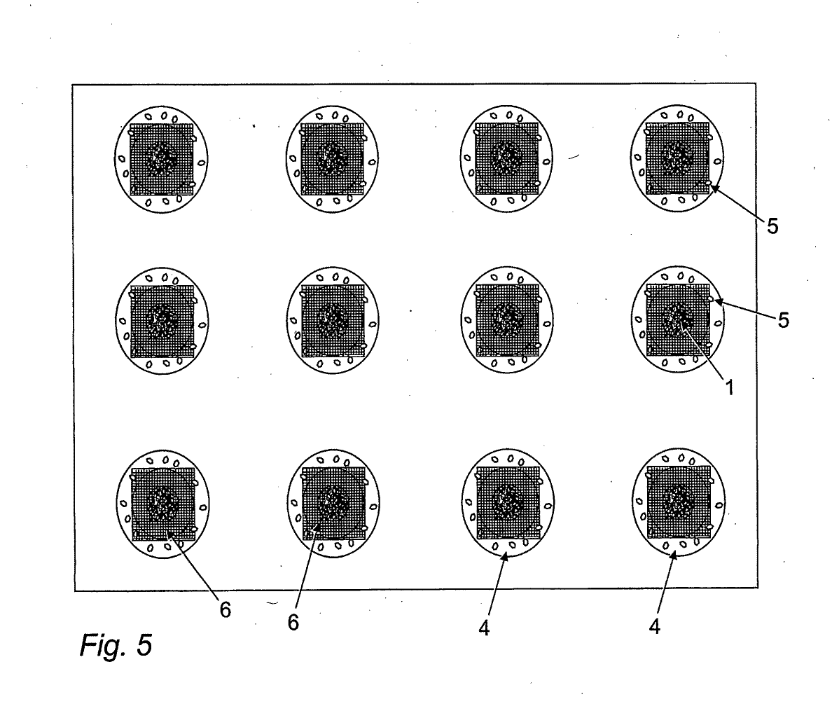

FIG. 5 illustrates an embodiment of the present invention using a 12 well-tissue culture plate wherein each vessel of the tissue culture plate includes a cell receiving zone and an agent receiving zone and tissue culture media has been supplemented with different treatments in each well for a defined period of time;

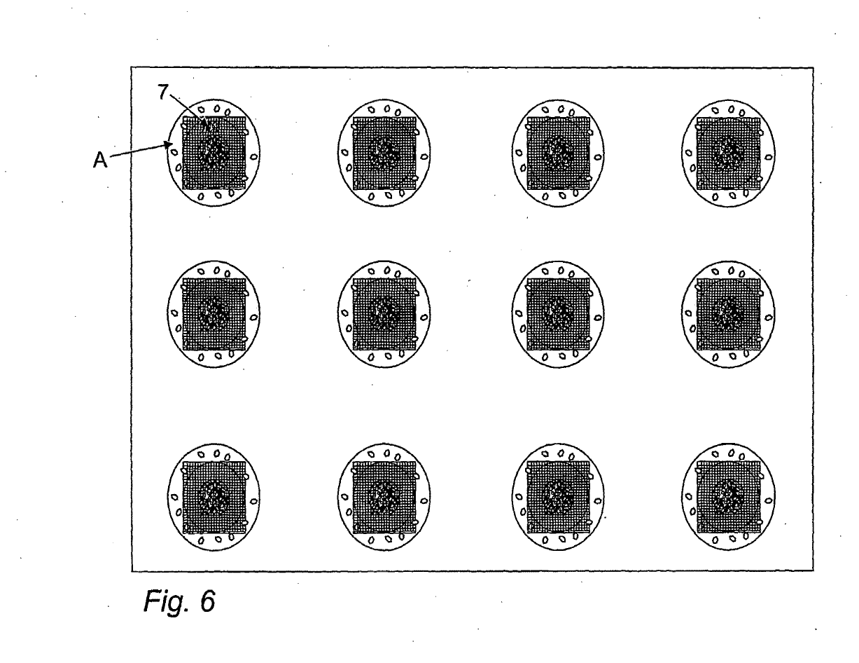

FIG. 6 is an illustration of an embodiment of the device of FIG. 5 showing a particular treatment which induces cell motility in at least a population of cells of the cell sample placed in the cell receiving zone wherein cell sample A indicates the treatment that induced the greatest motility; as determined by counting the squares covered in the grid as an objective measure of motility;

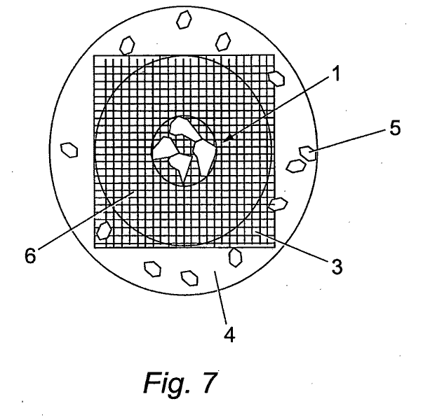

FIG. 7 is an illustration of an embodiment of the device of the present invention wherein four tissue explants (2 mm cubed in size) have been placed in the cell receiving zone (1);

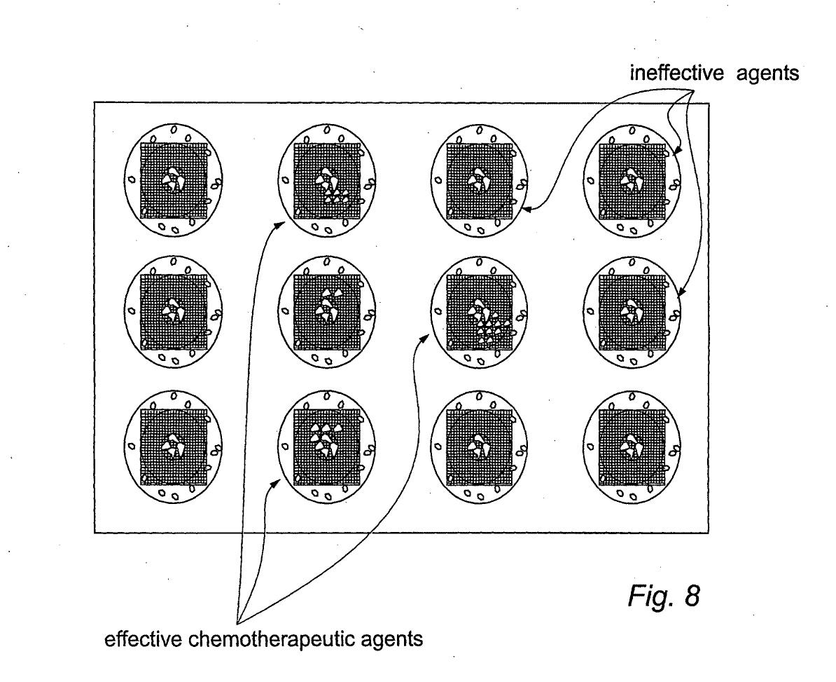

FIG. 8 illustrates an embodiment of the present invention wherein multiple cell receiving zones and agent receiving zones are arranged on a supporting substrate and a grid, located underneath the cell receiving surface of the supporting substrate allows measurement of the motility of cells of the tissue explants provided in the cell receiving zone;

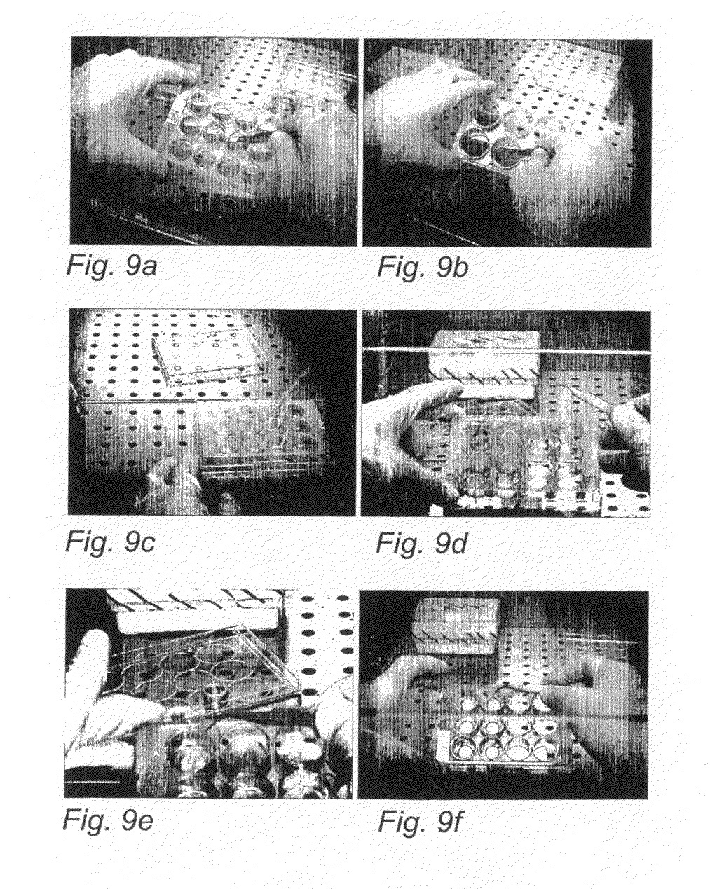

FIG. 9 shows an embodiment of the steps of method of the present invention;

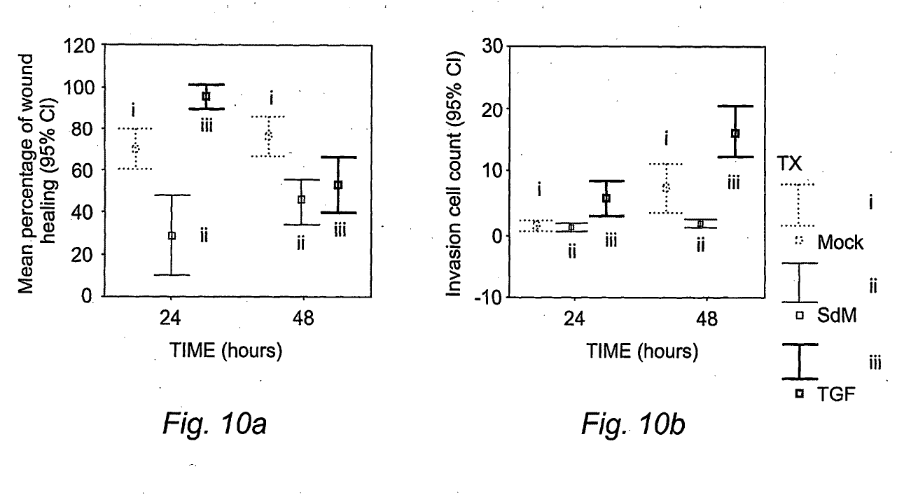

FIG. 10 details results of a conventional motility assay (A) and the Matrigel invasion assay (B);

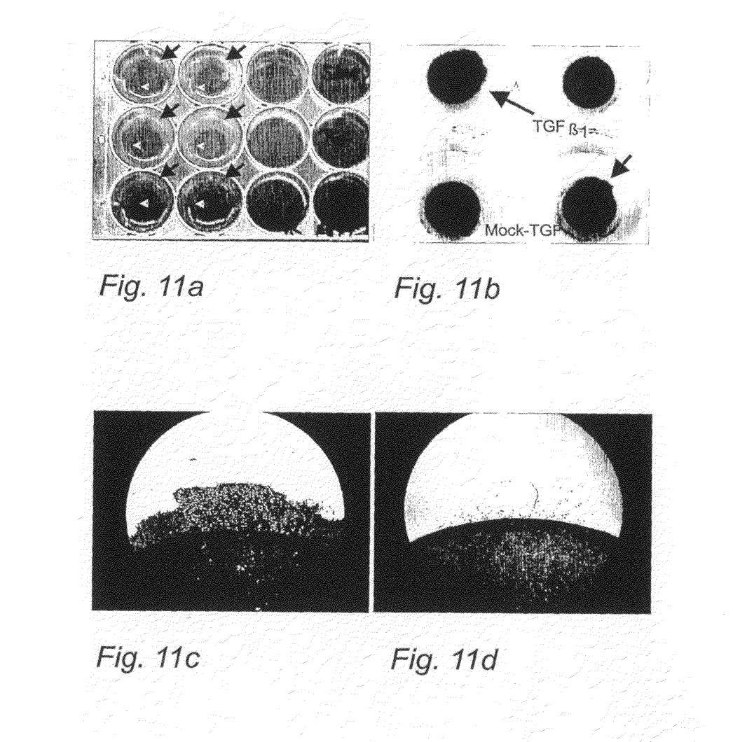

FIG. 11 shows the results using the novel invasion assay with agarose gel matrix (black arrow) and the centre coverslip containing AY-27 cells (white arrow head) which were bathed in SdM medium (A); and

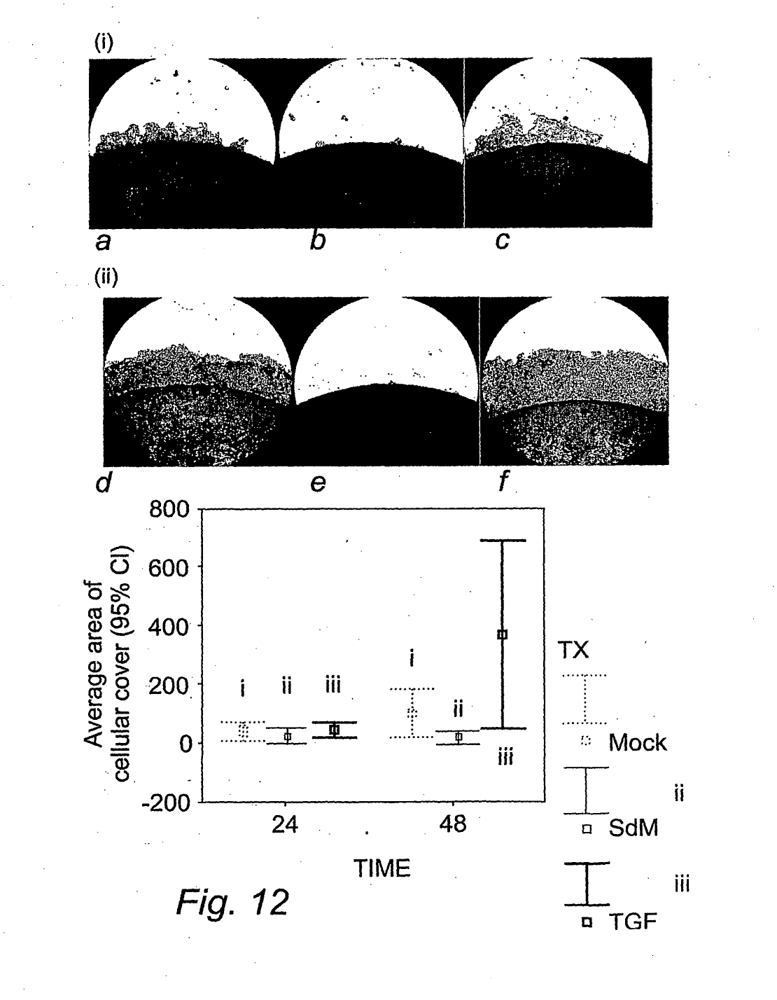

FIG. 12 (a) shows magnified digital images (×40) of Speedy-Diff Rapid Giemsa-stained AY-27 cells after treatment with control Mock-TGF (A & D), SdM (B & E) or TGF-β₁ (C & F) for 24 h (A,B,C) or 48 h (D,E,F).

### EXAMPLE

A comparison of the assay of the present invention with the scratch wound and Matrigel assays in the AY-27 bladder cancer cell line following treatment of transforming growth factor beta-1 (TGF-β₁), a known promoter to tumour progression was undertaken.

### Materials

Cell culture. The rat bladder cancer cell line AY-27 (a gift from Professor R. Moore at University of Alberta, Canada) was grown in 90% RPMI-1640 (Invitrogen), 10% Fetal Calf Serum (FCS) (Labtech International, Ringmer, East Sussex, England #4-101-500), 2% L-glutamine (Sigma Aldrich Co Ltd, Poole, Dorset England, #G6784) and 0.2% penicillin/streptomycin at 37° C. in a humidified CO₂ incubator (Shel Lab) with 95% oxygen and 5% carbon dioxide.

Reagents. Serum deprived medium (SdM) comprised RPMI-1640 with 0.002% FCS and 2% L-Glutamine. Recombinant human TGF-β₁ (R&D systems, Minneapolis, Minn., #240-B) was made into a 2 μg/ml stock suspension using the manufacturer's recommended diluents 1% Bovine Serum Albumin (BSA) and 0.004 mM Hydrochloric acid (HCL). Mock-TGF which acted as a control treatment comprised the diluents of TGF-β₁ in SdM.

### Conventional Scratch Wound Assay

Sub-confluent AY-27 cells were detached and 5×10⁶ were seeded into 6-well tissue culture plates. The cells were grown to 100% confluency before treatment with Mock-TGF, SdM or 3 ng/ml TGF-β₁ in duplicates for 24 h and 48 h. An in vitro “scratch wound” was created by scraping the confluent cell monolayer with a sterile pipette tip to make an approximate 1.0 mm gap. Three fixed points were marked in each well and phase-contrast images were captured at these same points over the 24 h and 48 h treatment course. The area of cells that had migrated beyond the ‘wound’ was estimated by superimposing a 5 mm×5 mm grid over the ‘wound’, and this was expressed as percentage when divided with the area of the ‘wound’.

### Conventional Matrigel Invasion Assay

FCS (10%) in 0.75 ml RPMI-1640 acted as the chemo-attractant. This was added to the lower compartment of 24-well BioCoat Matrigel invasion chambers (Becton Dickinson Labware, Bedford, Mass.) with an 8-μm pore polycarbonate filter coated with Matrigel. In the upper compartment, 8×10⁵ cells/well in SdM were placed in triplicate wells and incubated for 24 h and 48 h at 37° C. in a humidified incubator with 5% CO₂. After incubation, the cells that had passed through the filter into the lower wells were stained with Speedy-Diff Rapid Giemsa (Clin-Tech Ltd, Essex, UK. #61078, #61079, #61080) and counted by assessing 10 random high power fields images using ×400 magnification on a Leitz Labrodux K (Germany) light microscope.

### Assay of Present Invention

FIG. 9 illustrates the steps of the method of the present invention.

Ten millimetre coverslips were seeded with AY-27 cells at a density of 5×10⁵ and grown to 100% confluency in 12-well plates. The AY-27 cells at 100% confluency on 10 mm coverslips were removed (A) and washed vigorously using SdM medium (B).

In a separate 12-well culture plate, 1 ml of heated 2% agarose matrix consisting of 50% FCS and 50% supplemented RPMI thoroughly mixed was pipetted into each well, and allowed to cool and solidify (C).

A sterile 12 mm diameter metal ring was then used to cut out a circular area in the centre of the matrix (D & E) to provide a cell receiving zone and also to expose a portion of the supporting substrate to form the bridging portion between the cell receiving zone and the agent receiving matrix.

AY-27 cells on the 10 mm coverslip were then placed into the empty tissue receiving zone from which the agarose had been removed (F) such that the sample is spaced apart from and surrounded by the agarose (chemo-attractant agent receiving matrix).

An indentation provided in the cell receiving zone, allows reproducible locating of the coverslip including the cells to be tested in the cell receiving zone. The indentation is of suitable depth such that the top surface of the located slide is substantially flush with surrounding surface of the receiving surface. One millilitre of SdM alone or supplemented with 3 ng/ml TGF-β₁ or Mock-TGF was placed in the well for 24 h or 48 h. At the end of the time-points, the agarose matrix was removed and the cells were stained with Speedy-Diff Rapid Giemsa technique.

### Results

FIG. 10 details results of a conventional motility assay (A) and the Matrigel invasion assay (B). The error bar chart (A) shows results of a conventional cell motility assay wherein a wound of approximately 1 mm was created in 100% confluent AY-27 cells using a sterile pipette tip. Cells were treated with Mock-TGF (i), SdM (ii) or 3 ng.ml TGF-β₁ (iii) and images were captured after 24 h and 48 h. The percentage of wound healing was calculated by counting the number of boxes at least half covered by cells in the 5 mm×5 mm grid-rule (A). The error bar chart (B) shows results from a matrigel invasion assay wherein AY-27 cells in SdM were seeded into the upper chamber of the matrigel invasion assay. Cells were treated with Mock-TGF (i), SdM (ii) or TGF-β₁ (iii) and cells that passed successfully through the matrigel membrane after 24 h and after 48 h were Giemsa stained and enumerated. Significantly more cells were present in the membrane after TGF-β₁ treatment (Student's t-test, p<0.05) as illustrated by the distinct differences between the 95% confidence limit error bars (B);

FIG. 11 shows the results using the assay of the present invention with agarose gel matrix (black arrow) and the centre coverslip containing AY-27 cells (white arrow head) which were bathed in SdM medium (A). The gel matrix and bathing media were removed, and the contents of the well were stained with Speedy-Diff Rapid Giemsa to highlight the cell movement (B). Magnified digital images of TGF-β₁ treated AY-27 migrating towards the chemo-attractant (2% agarose gel containing RPMI supplemented with 50% FCS) (C) were captured. When the content of the 2% agarose gel matrix was made up with PBS alone or with RPMI-1640 growth medium in the absence of FCS, the cells did not migrate from the coverslip after 48 h (D) (×40 magnification, Leitz Wetsler microscope, Germany; Sony DSC-P92 digital camera, Japan); and

FIG. 12 (a) shows magnified digital images (×40) of Speedy-Diff Rapid Giemsa-stained AY-27 cells after treatment with control Mock-TGF (A & D), SdM (B & E) or TGF-β₁ (C & F) for 24 h (A,B,C) or 48 h (D,E,F). The results illustrate that 24 h after treatment, some cells in the control Mock-TGF (A) and SdM treated (B) wells had moved off the coverslip onto the supporting substrate and many cells had moved in the wells treated with TGF-β₁ (C). There were no significant differences in the numbers of migrating cells 24 h after the different treatments. However after 48 h, in comparison to Mock-TGF (D) or SdM (E) treated wells, significantly more cells had migrated following treatment with TGF-β₁ (F) (student t-test, p<0.05) as calculated by counting the number of boxes in the grid that were either completed covered or more the 50% covered with cells.

### TGF-β₁ Promotes Invasion in AY-27 Cells

TGF-β₁ treated cells had greater motility as demonstrated by an earlier wound closure at 24 h compared to the control treatment with SdM and Mock-TGF (FIG. 10A). However this effect did not persist to 48 h, possibly due to increased apoptosis because there was no chemo-attractant. Using the matrigel invasion assay, significantly more TGF-β₁ treated cells migrated across the matrigel (FIG. 10B) compared to the control groups at both time points (p<0.05, Student's t-test).

### Motility Assay

Preliminary studies demonstrated that AY-27 cells did not migrate from the coverslip (cell receiving zone) towards the agar (agent receiving matrix) when the agar that was not supplemented with FCS. When RPMI-1640 media supplemented with 50% FCS were added into the gel matrix, cells migrated across successfully (FIG. 11). Following treatment for 48 h significantly greater numbers of TGF-β₁ treated cells had migrated towards the agar in the novel assay in comparison to those treated with Mock TGF or incubated in SdM (FIG. 12)

The addition of 2% agarose maintained a solid nature to the chemo-attractant thereby preventing mixture of the chemo-attractant with the bathing serum deprived medium.

Various modifications and variations to the described embodiments of the invention will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention.

1. A device for measuring cell motility comprising a supporting substrate which includes at least one cell receiving zone and an agent receiving matrix provided on a matrix receiving zone, wherein interposed between the cell receiving zone and the agent receiving matrix is a bridging portion.

2. A device as claimed in claim 1 wherein the agent receiving matrix substantially surrounds the cell receiving zone.

3. A device as claimed in claim 1 wherein the supporting substrate is a cell culture plate comprising a plurality of wells wherein the base of each well comprises at least one cell receiving zone and an agent receiving matrix provided on a matrix receiving zone, wherein interposed between the cell receiving zone and the agent receiving matrix is a bridging portion.

4. A device as claimed in claim 1 wherein the cell receiving zone is of width in the range 5 mm to 20 mm, the matrix receiving zone is of width in the range 5 mm to 20 mm and the bridging portion is of width in the range 2 mm to 10 mm.

5. A device as claimed in claim 1 wherein the cell receiving zone is of width 10 mm, the matrix receiving zone is of width 10 mm and the bridging portion is of width 2 mm.

6. A device as claimed in claim 1 wherein the cell receiving zone is a portion of the supporting substrate which has an indentation adapted to receive cell support means on which a sample can be grown and/or mounted such that the sample can be provided to the device.

7. A device as claimed in claim 6 wherein the indentation in the supporting substrate is shaped and sized to locate a cell support means such that a top surface of the cell support means, when the cell support means is located in the indentation, is substantially in the same plane as the receiving surface of the cell receiving zone around the cell support means.

8. A device as claimed in claim 1 wherein the device comprises means of measuring movement of a cell from the cell receiving zone towards the matrix receiving zone.

9. A device as claimed in claim 8 wherein the means of measuring movement of a cell from the cell receiving zone towards the matrix receiving zone is a marking located on the supporting substrate.

10. A system for determining cell motility comprising:
i) a device comprising a supporting substrate which includes at least one cell receiving zone and a matrix receiving zone, wherein interposed between the cell receiving zone and the matrix receiving zone is a bridging portion; and
ii) an agent receiving matrix.

11. A system as claimed in claim 10 further comprising imaging means to allow detection of motility of cells provided to the device.

12. The system of claim 10 wherein the system further comprises a robotic device for fluid handling.

13. A kit for forming the device according to claim 1, said kit comprising:
a supporting substrate which includes at least one cell receiving zone and a matrix receiving zone, wherein interposed between the cell receiving zone and the matrix receiving zone is a bridging portion; and
an agent receiving matrix forming mould.

14. A kit as claimed in claim 13 wherein the mould is circular in shape and comprises one circumferential sidewall which defines the perimeter of the matrix to be formed.

15. A kit as claimed in claim 13 further comprising a cell receiving zone cut out member, wherein said member has a matrix cutting edge which, in use, can be placed into a formed matrix to cut a portion of the matrix and allow removal of said portion.

16. A kit as claimed in claim 15 wherein the cut out member has a diameter in the range 5 mm to 25 mm.

17. A kit as claimed in claim 13 wherein the mould comprises a first sidewall, a second sidewall and a matrix receiving channel interposed between said sidewalls.

18. An assay method using a device of claim 1 or a system of claim 10 comprising the steps:
providing a sample of cells to a cell receiving zone, such that the sample is in fluid connection with the agent receiving matrix, and
determining movement of cells provided in the sample from the cell receiving zone towards the agent receiving matrix.

19. An assay method as claimed in claim 18 further comprising at least one additional step selected from:
providing an agent to the agent receiving matrix,
characterisation of motile cells moving away from the cell receiving zone,
isolation of motile cells moving away from the cell receiving zone,
fixation for immunocytochemistry of motile cells moving away from the cell receiving zone,
characterisation of non-motile cells which do not move away from the cell receiving zone,
isolation of non-motile cells which do not move away from the cell receiving zone, and
fixation for immunocytochemistry of non-motile cells which do not move away from the cell receiving zone.

20. A method of using the device as claimed in claim 1 or a system as claimed in claim 10 to determine the motility of a cell of an individual tumour of an individual patient comprising the steps:
providing an explant derived from tissue obtained from a cancer patient to a cell receiving zone, such that cells of the explant are in fluid connection with the agent receiving matrix, and
determining movement of cells provided in the sample from the cell receiving zone towards the agent receiving matrix.

21. The method as claimed in claim 20 further comprising a step of: determining the ability of cells to express indicators of invasion potential.

22. A method to determine the ability of a test agent to modulate cell motility comprising the steps:
providing a sample of cells to a device according to claim 1 or a system according to claim 10 such that the sample is in fluid connection with the agent receiving matrix, and
determining movement of cells provided in the sample from the cell receiving zone towards the agent receiving matrix in the presence of a test agent in the matrix, relative to movement in the absence of said test agent.

23. The method as claimed in claim 22, wherein the movement of cells in the absence of said test agent is determined prior to the addition of test agent to the agent receiving matrix and determination of the movement of cells in the presence of said test agent.
